(19) United States
(12) Patent Application Publication
(10) Pub. No.: US 2026/0328872 A1
(43) Pub. Date: Oct. 22, 2026

(54) A Method and a System for Determining a Concentration of Magnetic Nanoparticles

(71) Applicant: THE UNIVERSITY OF BIRMINGHAM, Birmingham (GB)

(72) Inventors: Robert MORRIS, Nottingham (GB); Michael NEWTON, Nottingham (GB); Johanna STAFFORD, Nottingham (GB); Hareklea MARKIDES, Nottingham (GB); Alicia EL HAJ, Nottingham (GB)

(21) Appl. No.: 19/099,592

(22) PCT Filed: Jul. 28, 2023
(86) PCT No.: PCT/GB2023/052010

§ 371 Date: Jan. 29, 2025

(30) Foreign Application Priority Data
Jul. 29, 2022 (GB) 2211138.9

(51) Int. Cl. G01R 33/12; G01N 33/543

(52) U.S. Cl. CPC G01R 33/1276; G01N 33/54326

(57) ABSTRACT

A system and a method for determining a concentration of magnetic nanoparticles is provided. The method comprises: placing a sample in proximity to a Magnetic Particle Spectrometer system, the Magnetic Particle Spectrometer system comprising a drive coil, a receiving coil;
wherein the sample comprises Magnetic Nanoparticles MNPs binded to at least one cell membrane of the assay, wherein the MNPs have a core diameter d_{c};
transmitting a magnetic field H(t) having a sinusoidally varying magnitude via a drive signal in the drive coil, and measuring a response magnetisation M(t) of the MNPs via an induced emf in the receiving coil; determining a concentration c of the MNPs in the sample, by fitting a Langevin function to M(t) as a function of H(t).

This invention relates to a method and a system for determining a concentration of magnetic nanoparticles (MNPs). In particular, the invention finds utility in relation to determining a concentration of MNPs in a sample to support protocol development, translation and drug screening applications.

### BACKGROUND

Magnetic Nanoparticles (MNPs) are nanoparticles which can be manipulated using magnetic fields. Superparamagnetic Iron Oxide Nanoparticles (SPIONs) have found application in the development of cell based therapies and next generation dynamic drug screening To use MNPs/SPIONs for such applications amongst others, there is a need to quantify the MNPs/SPIONs in biological samples to provide accurate results for the given application. Current methods such as Inductively Coupled Plasma and the ferrozine assay are in routine use for quantisation of SPIONs, but these methods are slow, laborious and require destruction of the sample containing the SPIONs, which is unsuitable for some applications of SPIONs or MNPs.

### BRIEF SUMMARY OF THE DISCLOSURE

In devising the present invention, it has been realised that there is a need to find a method of quantifying MNPs in samples, including assays, which is not destructive, quick, low-cost and requires minimal understanding of the underlying technique broadening the possible range of users. It was realised by the inventors that Magnetic Particle Spectroscopy, a tool derived from Magnetic Particle Imaging, could provide a method of quantifying MNPs in a sample. In Magnetic Particle Spectroscopy, the MNPs align with an applied alternating magnetic field resulting in a flipping of the bulk magnetisation of the sample. The change in direction of the MNPs induces a signal in an appropriately placed receive coil which is amplified and analysed to reveal the properties of the sample.

Thus disclosed herein are methods and systems for using a Magnetic Particle Spectrometer system to determine the concentration of MNPs in a sample. As will be described below, the process for determining the concentration involves transmitting a sinusoidally varying magnetic field H(t) to a sample containing MNPs and measuring a response magnetisation M(t) of the MNPs. It was realised in the devising of this invention that by fitting a Langevin function to the collected data of the variation of M(t) with respect to H(t) that this could be used as a basis for measurement of the concentration of the MNPs in the sample. It was also realised in the devising of this invention that this process could be automated to find the concentration, and that calibrating the measurements could also be automated, thereby providing a method and system which is quick to use, does not require knowledge of the underlying technique and can provide a measurement of the concentration without destroying the sample.

Thus, viewed from one aspect, the present invention provides a method comprising: placing a sample in proximity to a Magnetic Particle Spectrometer system, the Magnetic Particle Spectrometer system comprising a drive coil, a receiving coil. The sample comprises Magnetic Nanoparticles (MNPs) binded to at least one cell membrane of the sample, wherein the MNPs have a core diameter d_{c}. The method further comprises transmitting a magnetic field H(t) having a sinusoidally varying magnitude via a drive signal in the drive coil, and measuring a response magnetisation M(t) of the MNPs via an induced emf in the receiving coil. The method further comprises determining a concentration c of the MNPs in the sample, by fitting a Langevin function to M(t) as a function of H(t).

In accordance with the present invention, the concentration of the MNPs in the sample can be determined without destroying the sample.

In embodiments, the Langevin function is:

$$M ( t ) = {m}_{s} c ( coth [ \frac{{m}_{s} {μ}_{0} H ( t )}{{k}_{B} T} ] - \frac{{k}_{B} T}{{m}_{s} {μ}_{0} H ( t )} ) , {m}_{s} = \frac{1}{6} π {d}_{c}^{3} {M}_{s} ,$$

where μ₀ is the permeability of free space, k_{B} is the Boltzmann constant and Mₛ is a known saturation magnetisation of the MNPs at a temperature T.

In embodiments, the method comprises: determining the temperature T proximal to the Magnetic Particle Spectrometer system whilst the magnetic field H(t) is transmitted. This process is therefore sensitive to the environment it is conducted in.

In embodiments, the method comprises comparing the determined concentration c against a concentration threshold to verify the sample. This process can therefore be quickly carried out to verify the concentration of the MNPs, to ensure that the sample contains the necessary uptake of MNPs to fulfil the necessary requirements for its application.

According to embodiments, the Magnetic Particle Spectrometer comprises a cancellation coil, arranged to cancel out an induced emf in the receiving coil due to the drive signal. The method therefore provides a measurement of the concentration which is based purely on the emf induced by the MNPs.

According to embodiments, the method comprises: determining the concentration c of MNPs in multiple such samples and determining the variation in concentration c between the multiple samples. The method therefore can determine batch to batch consistency for samples which are directed to the same application. For example, where each sample comprises an assay, the method therefore can determine batch to batch consistency for several such assays.

According to embodiments, the drive signal is provided at less than 1 kHz. According to embodiments, the drive signal is provided at less than 500 Hz; optionally the drive signal is provided at around 300 Hz. Optionally, the drive signal is provided at 100 Hz. Therefore this provides a method which does not require expensive high frequency components.

According to embodiments, the MNPs in the sample are Superparamagnetic Iron Oxide Nanoparticles.

According to embodiments, the sample comprises an assay.

Viewed from another aspect, the present invention provides a Magnetic Particle Spectrometer system comprising:
a drive coil, a receiving coil;
the system is associated with a sample comprising Magnetic Nanoparticles (MNPs) binded to regions of at least one cell membrane of the sample, wherein the MNPs have a core diameter d_{c}. The sample is located proximal to the Magnetic Particle Spectrometer system. The system is arranged to:
transmit a magnetic field H(t) having a sinusoidally varying magnitude via a drive signal in the drive coil, and measure the response magnetisation M(t) of the MNPs via an induced emf in the receiving coil. The system is arranged to determine a concentration c of the MNPs in the sample, using a Langevin function.

In accordance with the present invention, the concentration of the MNPs in the sample can be determined without destroying the sample.

According to embodiments, the Langevin function is:

$$M ( t ) = {m}_{s} c ( coth [ \frac{{m}_{s} {μ}_{0} H ( t )}{{k}_{B} T} ] - \frac{{k}_{B} T}{{m}_{s} {μ}_{0} H ( t )} ) , {m}_{s} = \frac{1}{6} π {d}_{c}^{3} {M}_{s} ,$$

where μ₀ is the permeability of free space, k_{B} is the Boltzmann constant and Mₛ is a known saturation magnetisation of the MNPs at a temperature T.

According to embodiments, the system is arranged to determine the temperature T proximal to the Magnetic Particle Spectrometer system whilst the magnetic field H(t) is transmitted. The system therefore provides further automation by measuring the temperature T to determine the concentration, and the system is sensitive to the environment.

According to embodiments, the Magnetic Particle Spectrometer system comprises a cancellation coil, arranged to cancel out an induced emf in the receiving coil due to the drive signal.

According to embodiments, the cancellation coil comprises a threaded surface, and the receiving coil comprises a threaded surface arranged to receive the cancellation coil via a threaded connection, wherein the position of the cancellation coil relative to the receiving coil is adjustable by rotating the cancellation coil relative to the receiving coil. According to embodiments, the system is arranged to automatically adjust the position of the cancellation coil relative to the receiving coil using a closed feedback loop to optimally cancel out the induced emf in the receiving coil due to the drive signal. The system therefore can automate calibration to reduce user input to obtain the optimal measurement of the concentration. The system can therefore be operated by a broad range of users, as the underlying technique does not need to be known.

According to embodiments, the drive signal is provided at less than 1 kHz. According to embodiments, the drive signal is provided at less than 500 Hz; optionally the drive signal is provided at around 300 Hz. Optionally, the drive signal is provided at 100 Hz. Therefore this provides a system which does not require expensive high frequency components.

According to embodiments, the MNPs in the sample of the system are Superparamagnetic Iron Oxide Nanoparticles.

According to embodiments, the sample comprises an assay.

### BRIEF DESCRIPTION OF THE DRAWINGS

Embodiments of the invention are further described hereinafter with reference to the accompanying drawings, in which:

FIG. 1A shows a graph illustrating an example of how the strength of the magnetic field H(t) transmitted via a drive signal from a drive coil of the Magnetic Particle Spectrometer system varies over time;

FIG. 1B shows a graph illustrating an example of how the response magnetisation M(t) of the MNPs varies over time;

FIG. 1C shows a graph illustrating an example of how the magnetisation M(t) of the sample of MNPs varies according to the strength of the magnetic field H(t);

FIG. 2A shows a schematic of an example Magnetic Particle Spectrometer system;

FIG. 2B shows part of an example Magnetic Particle Spectrometer system;

FIG. 3 shows part of an example Magnetic Particle Spectrometer system, in particular a cross-section of a part of the Magnetic Particle Spectrometer system;

FIG. 4 shows part of an example Magnetic Particle Spectrometer system, in particular an exploded view of part of a Magnetic Particle Spectrometer system;

FIG. 5 shows a graph illustrating how the measured signal from a receiving coil varies according to an applied field in a drive coil;

FIG. 6 shows a graph illustrating an example of a calibration series and validation of detection of MNPs in cells;

FIG. 7 shows a graph illustrating an example method;

FIG. 8 is a photograph showing the position of a thermocouple to measure the temperature of the system and sample;

FIG. 9 is a plot showing the ability to measure MPS signals at different frequencies of interest;

FIG. 10 shows a photograph of the automated positioning system for the cancellation coil; and

FIG. 11 shows samples which are above and below a nominal rejection threshold for nanoparticle concentration as measured with the MPS device

### DETAILED DESCRIPTION

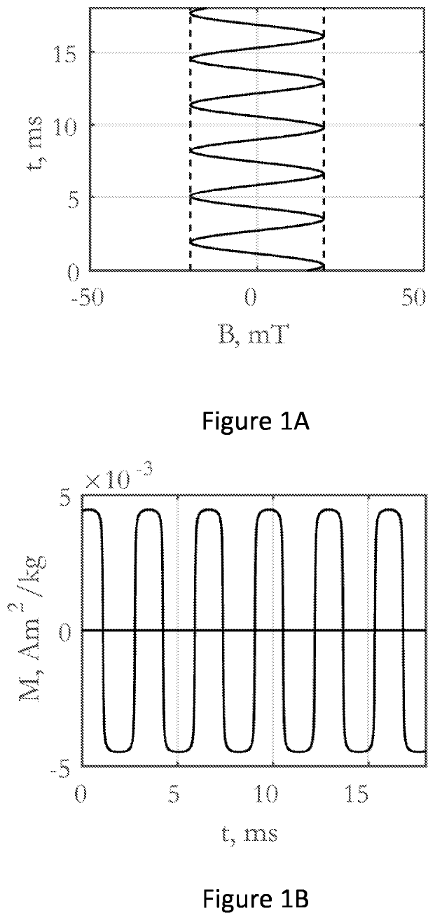

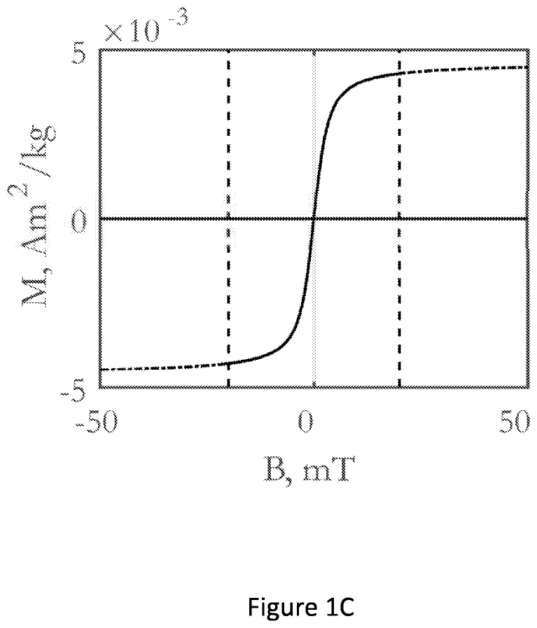

FIG. 1A illustrates a graph showing an example of how a field strength B of a magnetic field H(t) transmitted via a drive signal from a drive coil of a Magnetic Particle Spectrometer system varies over time. As shown in FIG. 1A, the field strength B varies sinusoidally between a maximum positive value and a minimum negative value. FIG. 1B illustrates a response magnetization M(t) that occurs in the receive coil due to the magnetic field H(t). In the presence of Magnetic Nanoparticles (MNPs) such as Superparamagnetic Iron Oxide Nanoparticles (SPIONs), the waves of M(t) become more square, as can be appreciated. The Electromotive Force (EMF) induced in the receive coil of the Magnetic Particle Spectrometer as a result of this signal, can be used as discussed below to determine the concentration of MNPs in the sample. FIG. 1C illustrates how the magnetization M(t) varies with respect to the strength B of the magnetic field H(t), this is typically called a magnetization curve. It shows that the magnetization sharply rises at first but then levels out both for the positive and negative magnitude of the strength B. The levelling out is due to a majority of the MNPs being aligned with the field H(t), and no further increase of the field strength B will substantially alter the magnetization response of the MNPs.

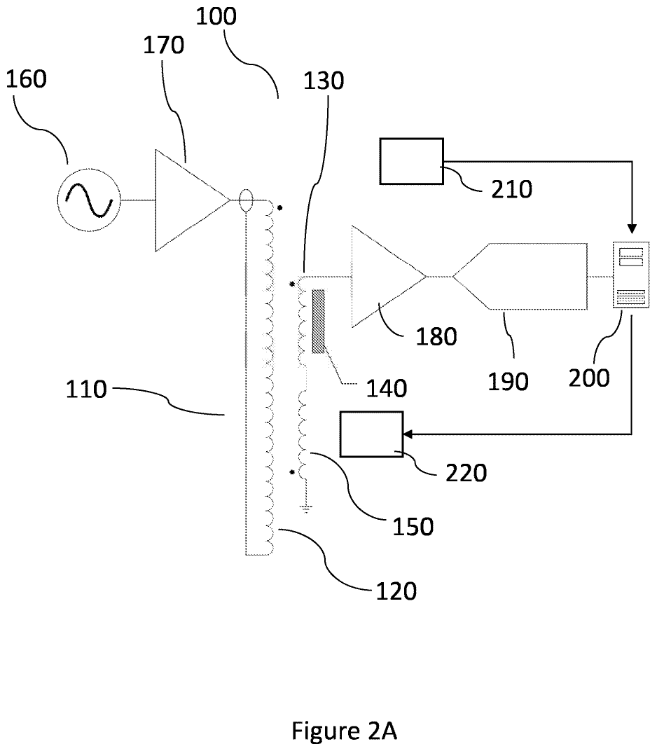

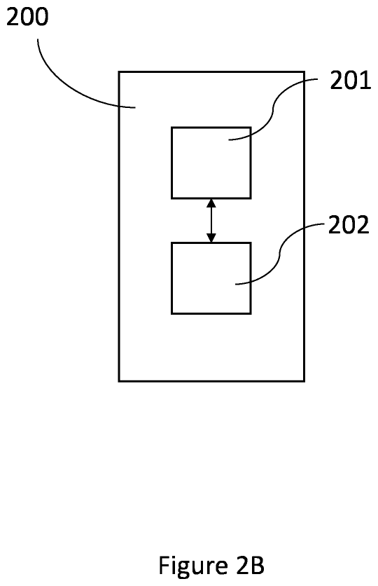

FIG. 2A illustrates an example Magnetic Particle Spectrometer system 100. The system 100 comprises a set of conductive coils 110. The conductive coils 110 comprises a drive coil 120, a receiving coil 130. The system 100 is also associated with a sample 140. The sample 140 comprises magnetic nanoparticles (MNPs) which have a core diameter d_{c}. The MNPs can be Superparamagnetic Iron Oxide Nanoparticles (SPIONs). The sample 140 is located proximal to the Magnetic Particle Spectrometer system 100. In the example of FIG. 2A, the sample 140 is located within the set of conductive coils 110. The set of conductive coils 110 can comprise an aperture such as a bore which the sample 140 is located within in use.

The system 100 is arranged to: transmit a magnetic field H(t) having a sinusoidally varying magnitude via a drive signal in the drive coil, and measure the response magnetisation M(t) of the MNPs via an induced emf in the receiving coil 130. From the resulting induced EMF, the system 100 is arranged to determine a concentration c of the MNPs in the sample 140 using a Langevin function.

As illustrated in FIG. 2A, the system 100 also comprises: a cancellation coil 150; a signal source 160 which in this example provides the sinusoidally varying signal which is used to transmit the sinusoidally varying magnetic field H(t); a power amplifier 170 which amplifies the signal from the signal source 160; a small signal amplifier 180, which amplifies the signal received from the receiving coil 130 and cancellation coil 150 combination; an analogue to digital converter (ADC) 190 and a data acquisition device, computing device or computer 200. The data acquisition device, computing device or computer 200 comprises one or more processor(s) 201 and memory 202, as illustrated in FIG. 2B. In some examples, the signal source 160 provides the drive signal at less than 3 kHz. In some examples, the drive signal is provided at less than 1 kHz. In some examples, the drive signal is provided at less than 500 Hz. In some examples the drive signal is provided at around 300 Hz. In some examples, the drive signal is provided at 100 Hz.

The system 100 can also comprise a temperature sensor 210 arranged to provide temperature data representing a temperature reading to the computer 200 and a motor 220 arranged to change and control the position of the cancellation coil 150 relative to the receiving coil 130. The motor 220 is controlled by the computer 200.

As an example, the drive coil 110 is made of a litz wire (81/0.04 mm) which is formed into the drive coil (70 mm Ø×102 mm) comprising of 6 layers, connected as 3 parallel pairs of windings. The power amplifier 170 is a TA2400 amplifier, which receives the signal from a signal generator (signal source 160) operating at 300 Hz and 0.5v peak to peak voltage (AFG-2225) for a total final drive power of 65 W. The receiving coil 130 and cancellation coil 150 (each 11 mm Ø×14 mm) are wound using similar litz wire around 3D printed bobbins using a standard clear resin (FLGPCL04, FormLabs, MA, USA) as it was found that some black resins contained magnetically active compounds which were not suitable for subsequent measurements. Thus resins free of magnetic material may be preferred. In this example, the sample 140 is placed within the receiving coil 130 and the signal received from the receiving coil 130 is then amplified by a pre-amplifier MPA-102 (small signal amplifier 180) and digitized by the first channel of a soundcard UMC202HD (analogue to digital converter 190). The second channel of the sound card is fed by a pickup coil mounted atop the system 100 to minimise coupling to the other coils but to facilitate capture of the drive signal as experienced by the receiving and cancellation coil (i.e. not simply the output of the power amplifier). The computer 200 collects periodically, such as 250 ms although other time interval may be used, blocks of these signals with a 192 kHz sampling frequency which are processed in MATLAB (Mathworks, MA, USA).

It was realised whilst devising the present invention that by collecting the magnetization data from the Magnetic Particle Spectrometer 100 when a sample 140 containing MNPs is provided in proximity to the Magnetic Particle Spectrometer system 100, the measurements could be used to determine the concentration of the MNPs in the sample 140. It is known from “Magnetic particle imaging: advancements and perspectives for real-time in vivo monitoring and image-guided therapy”, Michelle H. Pablico-Lansigan, Shu F. Situ and Anna Cristina S. Samia, Nanoscale, 2013, 5, 4040, that the magnetization of SPIONs in a Magnetic Particle Spectrometer follow the equation

$$M ( t ) = {m}_{s} c ( coth [ \frac{{m}_{s} {μ}_{0} H ( t )}{{k}_{B} T} ] - \frac{{k}_{B} T}{{m}_{s} {μ}_{0} H ( t )} ) , {m}_{s} = \frac{1}{6} π {d}_{c}^{3} {M}_{s}$$

where M(t) is the magnetisation as a function of time for a sample of concentration c, for particles with core diameter d_{c} and saturation magnetisation Mₛ at a temperature of T (in Kelvin), subjected to an applied field of H(t). μ₀ is the permeability of free space and k_{B} is Boltzmann's constant.

It was surprisingly realised by the inventors during the devising of the present invention that the above equation could be used to determine the concentration of the MNPs in the sample, if all other quantities are known. For characterised MNPs, the core diameter d_{c} and the saturation magnetisation Mₛ are known. Determining the temperature T leaves the only unknown quantity to be concentration c.

Using the system 100, the digitised blocks of data collected as described above, are similar to the B and H signals of a traditional B-H curve. The near continuous distribution of measured values equivalent to B are quantised into finite values, allowing averaging of the values equivalent to H. This produces a Langevin response plot according to the equation above. A sum of squares minimisation can then be undertaken between this and the applied field averaged signal to determine the only unknown which is the sample concentration. Each data point is calculated from a plurality, such as 5 measurements (although other numbers may be used) to allow for error and hence confidence estimation. In contrast to the more traditional power spectral density to harmonic amplitude approach, where much of the signal is discarded in unused harmonic signals, this approach utilises every collected point in the calculation and is therefore less susceptible to the influence of noise, particularly at lower iron concentrations.

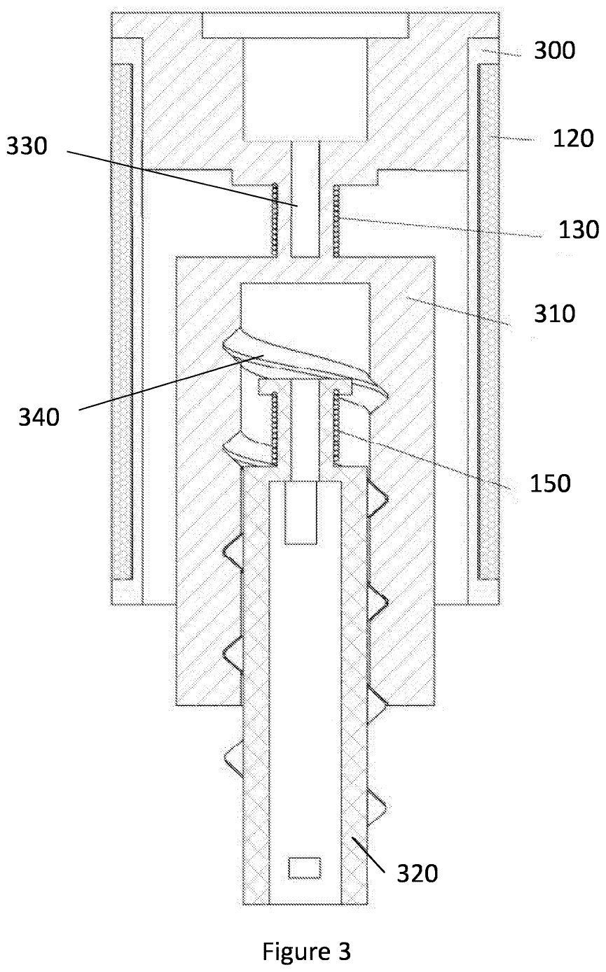

FIG. 3 shows a cross section of a set of conductive coils 110 according to the present invention. One of the advantages of the present invention is its ease of use for a person who does not have a high level of knowledge of the underlying techniques of the Magnetic Particle Spectrometer. To provide this advantage, much of the process of collecting the magnetization data from the sample is automated, which also provides a system which is reliable with its measurements. To achieve this advantage, the drive coil 120 and the receiving coil 130 may each comprise secure supports so that their position relative to one another is not changed and remains fixed. The drive coil comprises a drive coil support 300 and the receiving coil comprises a receiving coil support 310. The cancellation coil 150 may also comprise a cancellation coil support 320. In this example, the Magnetic Particle Spectrometer system 100 comprises a bore 330, in which the sample 140 can be placed in use. When the sample 140 is located in the bore, it is held within the receiving coil 130 remote from the cancellation coil 150.

As the receiving coil 130 is held inside the drive coil 120, the drive signal will also induce an emf in the receiving coil 130 which must somehow be removed from the resulting signal to allow the signal due to the induced emf from the MNPs to be seen which is magnitudes smaller than the emf induced in receiving coil 130. To achieve this the cancellation coil 150 is used which cancels out the drive signal received in the receiving coil 130 so that the resulting signal is only that produced by the MNPs. The cancellation coil 150 may be identical to the receiving coil 130 but counter wound. The cancellation coil may be placed within the drive coil 120. It has been found that the position of the cancellation coil 150 with respect to the receiving coil 130 has a significant impact on the efficiency of the cancellation and therefore the success of the measurement. In this example, the position of the drive coil 120 and the receiving coil 130 is fixed by the drive coil support 300 and the receiving coil support 310. In other examples, the positions of the receiving coil 130 and the drive coil 120 can be changed relative to one another. For example, the receiving coil 130 may have a threaded connection with the drive coil 120 in a similar manner to the threaded connection between the cancellation coil 150 and the receiving coil 130 described below.

It has been identified that positioning the cancellation coil 150 for the optimal efficiency of the cancellation is a slow manual task and requires skill and knowledge of the underlying technique from the operator to achieve a good result. Embodiments of the present invention therefore comprise automatic positioning of the cancellation coil with respect to the drive coil, so that users without skill and knowledge of the underlying technique can use the Magnetic Particle Spectrometer system 100. In particular the cancellation coil 150 comprises a threaded surface 340. The receiving coil 130 comprises a corresponding threaded surface 400 arranged to receive the threaded surface 340 of the cancellation coil 150 by a threaded connection. The position of the cancellation coil 150 relative to the receiving coil 130 is adjustable by rotating the cancellation coil 150 relative to the receiving coil 130. In this example the threaded surface 340 is provided on the cancellation coil support 320.

The system 100 can be arranged to automatically just the position of the cancellation coil 150 relative to the receiving coil 130. The system 100 can use a closed feedback loop to optimally cancel out the induced emf in the receiving coil 130 due to the drive signal.

As an example to provide this automatic adjustment, the system 100 can comprise an actuator such as stepper motor (motor 220) which is arranged to adjust the position of the cancellation coil 150 by moving, such as rotating it, in small increments to find the position of which the emf induced in the receiving coil 130 due to the drive coil 120 is reduced to a minimum in the absence of a sample. This process can be automated by the computer with the one or more processor(s) 210 and memory 220, where the memory 220 can store computer program instructions, which when executed by the processor(s) 210, uses the closed feedback loop to change the position of the cancellation coil 150 until it detects the minimum drive signal picked up by the receiving coil 130 and cancellation coil 150 combination.

In other examples, the relative position of the cancellation coil 150 and the receiving coil 130 can be controlled in an alternate way to the threaded connection and the stepper motor. For example, the cancellation coil 130 can be slidingly inserted into the receiving coil 130 and the position of the cancellation coil 130 can be changed and controlled by a linear actuator causing sliding movement of the cancellation coil 150. The linear actuator can stop at predefined positions to control the position of the cancellation coil 150. One or more guides may be provided on cancellation coil 150 or the receiving coil 130 to control the positioning.

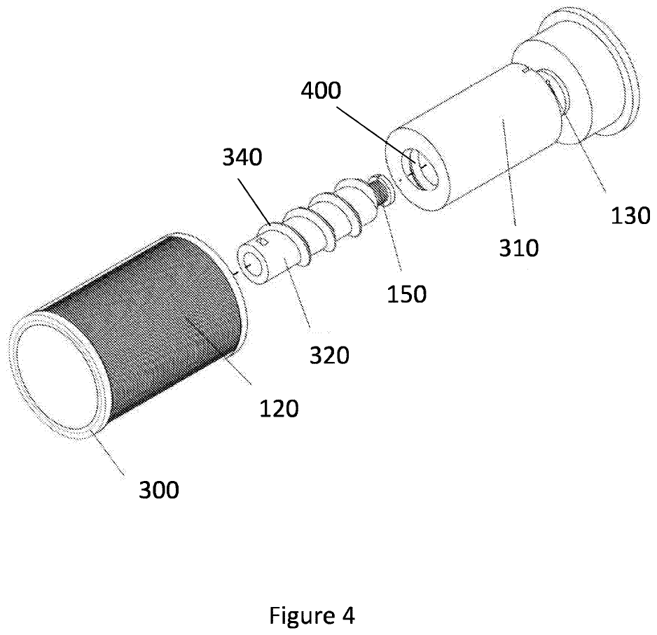

FIG. 4 shows an exploded view of an example set of coils 110, and in particular shows the threaded surface 340 of the cancellation coil 150 which corresponds in a threaded connection with a threaded surface 400 in the receiving coil 130. The drive coil 120 may fit over both the cancellation coil 150 and the receiving coil 130.

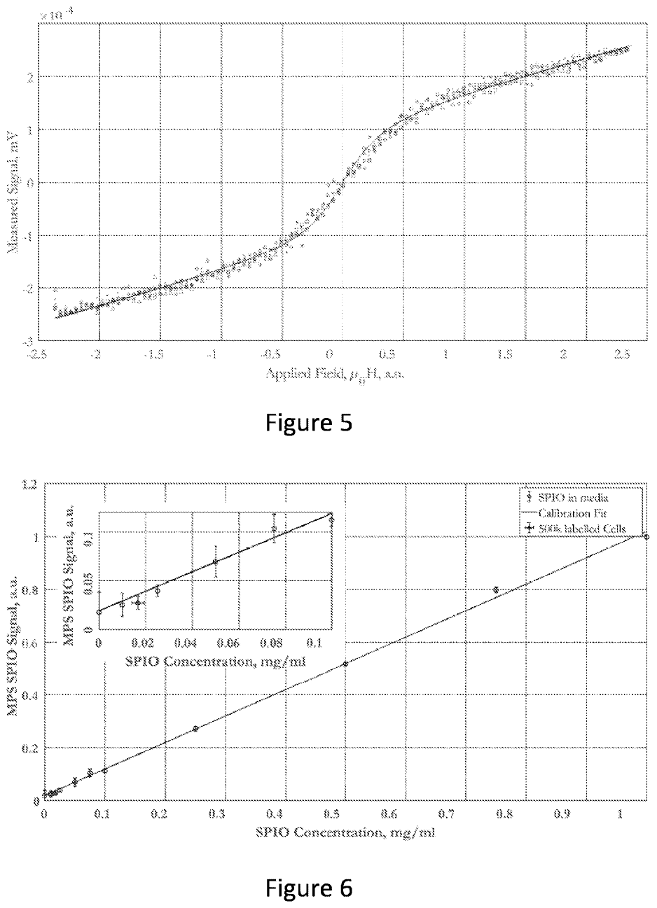

FIG. 5 shows a graph which illustrates how the measured signal from the receiving coil 130 of the emf induced due to the MNPs in the sample 140 varies according to the applied field H(t). Each data point is calculated from 5 such measurements in this example to allow for error and hence confidence estimation. The line of best fit shows the Langevin function which is fitted to find the concentration c.

To assess the effectiveness of the system 100, a calibration series was produced. The calibration series consisted of (0.01-1)mg/ml commercial SPIOs (Synomag-D, 70 nm, uncoated dextran surface, Micromod, Germany) resuspended in cell expansion media. The resulting MPS signal for each sample, plotted in an arbitrary unit, is shown in FIG. 6, labelled as “SPIO in media”, with a line of best fit providing the “Calibration fit”.

To assess the effectiveness of the system 100 in detecting and quantifying SPIONs within a biological system, 500,000 mesenchymal stem cells (MSCs) were labelled with similar SPIOs at a concentration of 10 mg/ml. Here, MSCs were initially cultured and expanded in basal media consisting of DMEM media supplemented with 10% foetal bovine serum (FBS), 1% L-Glutamine and 1% Penicillin Streptomycin and incubated at 37° C. and 5% CO2 until confluent. Cells were then harvested and plated within tissue culture flasks and allowed to attach overnight prior to the addition of the SPION labelling solution which consisted of Synomag SPIONs resuspended in serum free media (SFM) at a concentration of 10 mg/ml. To encourage optimal and efficient uptake of Synomag, a novel cell penetrating peptide, P21-8R [(Dixon et al., 2016, Markides et al., 2019)] was implemented. This was supplemented within the labelling solution at a concentration of 1 mM per 50 μg of SPIOs per 200,000 cells. Cells were finally harvested, and 500,000 cells pelleted and resuspended in 200 μl of expansion media for MPS detection. Three such samples were prepared and measured.

Once all tests of the three samples using the MPS had been completed, the cells were digested in concentrated hydrochloric acid whilst being sonicated for 10 minutes. The iron content of the samples was estimated using a potassium dichromate titration to oxidise the ferrous iron of the SPIOs in an acidic solution with barium diphenylamine sulphonate indicator until reaching a dark purple end point. The same process was repeated for the 10 μg/ml and 25 μg/ml samples to allow the results to be appropriately scaled to the SPIO concentration value. The resulting MPS signal is then plotted against the estimated iron concentration using a black (*) in FIG. 6, labelled as “500k labelled cells”. The portion of the graph inset in the top left of FIG. 6 shows a zoomed in view of the graph from 0-0.1 on the x-axis and 0 to ~1.2 on the y-axis, showing the location of the “500k labelled cells” plot.

The system 100 produces a linear response to iron concentration with good sensitivity demonstrated down to 10 μg/ml in 200 μl samples. Using the calibration fit as shown in FIG. 6, the iron concentration is estimated as (12±8)μg/ml by the calibration fit which is in good agreement with the titration estimate of (16±3)μg/ml shown by the black (*).

The sample 140 can be an assay 140. For example, the assay can be a cell-based assay, a non-cell based assay, a multiwell assay, an organ on a chip assay, amongst others. In one example the assay 140 is a DYNASCREEN assay (https://www.micabiosystems.co.uk/dynascreen). The DYNASCREEN is an assay where SPIONs are encouraged to precisely bind to certain regions of the cell membrane of CaCO₂ cells grown in monolayer. Once attached, the SPIONs respond to the application of an external magnetic field (delivered via custom designed magnetic force bioreactor) thereby producing deformations on the membrane at a frequency similar to gut peristaltic contractions. This effectively creates a dynamic environment allowing for the better prediction of in vivo drug permeability. The assay 140 can therefore comprise CaCO₂ cells grown in monolayer with SPIONs binded to certain regions of the cell membrane. The DYNASCREEN assay, prepared in transwells can be validated by extracting each transwell briefly from media and placing within the bore of the spectrometer. The system 100 can therefore advantageously verify the correct preparation of the DYNASCREEN or similar assays by determining the quantity and location of SPIONs. The system 100 therefore allows for verification of cellular labelling before the addition of the drug (for drug testing using DYNASCREEN) thus allowing for quality control.

As mentioned above, the Magnetic Nanoparticles (MNPs) can be Superparamagnetic Iron Oxide Nanoparticles. In other examples, MNPs can be particles comprising oxides or other compounds of magnetic elements such as iron, nickel or cobalt, or superparamagnetic nanoparticles which may comprise one or more of single crystal domain magnetite or maghemite or rare earth elements such as Dysprosium, Neodymium or Praseodymium.

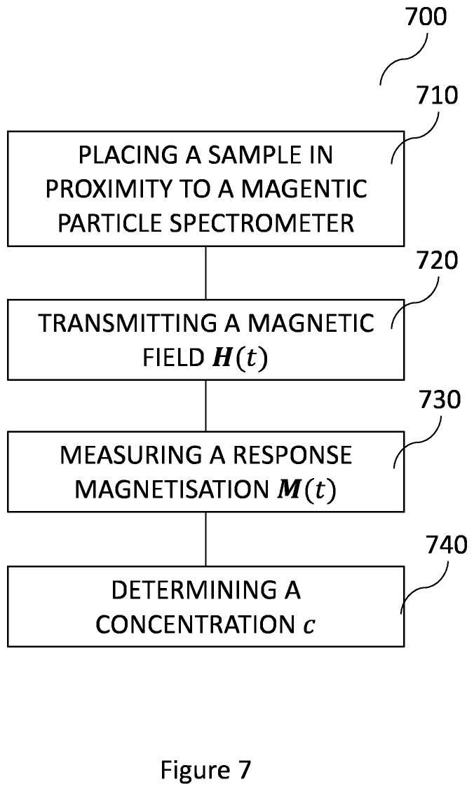

FIG. 7 illustrates an example method 700. The method comprises: placing 700 a sample in proximity to a Magnetic Particle Spectrometer system. The Magnetic Particle Spectrometer system comprising: a drive coil; a receiving coil. The sample comprises Magnetic Nanoparticles (MNP) binded to at least one cell membrane of the sample. The MPs having a core diameter d_{c}. The method 700 comprises at step 720, transmitting a magnetic field H(t)having a sinusoidally varying magnitude via a drive signal in the drive coil and then step 730 measuring a response magnetization M(t) of the MNPs via an induced emf in the receiving coil. The method 700 also comprises in step 740 determining a concentration c of the and MNPs in the sample, by fitting a Langevin function to the Magnetization M(t) as a function of H(t).

The Langevin function can be:

$$M ( t ) = {m}_{s} c ( coth [ \frac{{m}_{s} {μ}_{0} H ( t )}{{k}_{B} T} ] - \frac{{k}_{B} T}{{m}_{s} {μ}_{0} H ( t )} ) , {m}_{s} = \frac{1}{6} π {d}_{c}^{3} {M}_{s} ,$$

The method 700 can also comprise as a step: determining the temperature T proximal to the Magnetic Particle Spectrometer system whilst the magnetic field is transmitted.

The Magnetic Particle Spectrometer system can comprise a cancellation coil, arranged to cancel out an induced emf in the receiving coil due to the drive signal. Additional features of the Magnetic Particle Spectrometer system used to achieve the method can be as described above for the system 100.

The method 700 can additionally comprise comparing the determined concentration c against a concentration threshold to verify the sample. This is to determine whether the sample has had the required uptake of MNPs required for the sample to be used for its specific application.

The method can also comprise: determining the concentration of MNPs in multiple such samples and determining the variation in concentration between the multiple samples. This enables reliable comparison for batch to batch consistency of samples which are required for the same application. For example, when the samples each comprise an assay, this enables reliable comparison for batch to batch consistency of assays. For example multiple such assays for the DYNASCREEN assay can be prepared which have a consistent concentration of SPIONs, to enable different types of drug tests or repetitions the same drug test to be performed reliably.

The Magnetic Particle Spectrometer system 100 and methods described herein can also be used in-line with magnetic cell sorting devices e.g. https://www.miltenyibiotec.com/GB-en/products/macs-cell-separation.html, where it can be used as a real time monitor of the process.

Unlike other similar systems, it will be possible to obtain estimates of cellular label concentration with minimal understanding of the underlying technique broadening the possible range of users. The total cost of this Magnetic Particle Spectrometer system is low compared to other methods, which brings further desirability to adoption of the system in cell biological labs to quantify the uptake of MNPs. The system can be set up to have automated signal processing which can be used along with calibration to provide go, no-go indication for assay results of interest bringing with it the possibility of point of care assay biosensing.

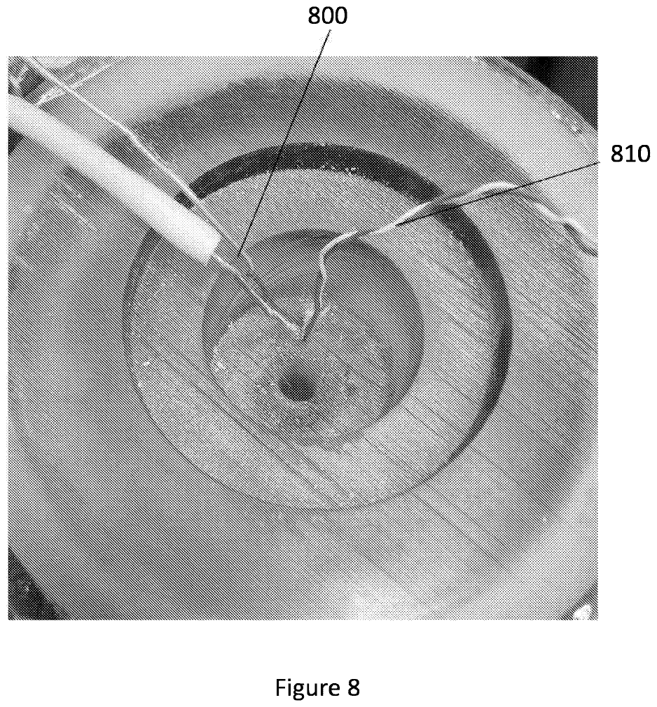

FIG. 8 shows a photograph of the use of a thermocouple to measure the temperature of the Magnetic Particle Spectrometer system 100 and sample 140 during a measurement to improve the accuracy of the Langevin fit. The receiving coil 130 is connected to the rest of the Magnetic Particle Spectrometer system 100 by wires 800. The pair of twisted thermocouple wires 810 is inserted proximal to the receiving coil 130. Temperature measurements are interleaved with the Magnetic Particle Spectrometer system 100 measurements to minimise interference between the Magnetic Particle Spectrometer system 100 and the thermocouple.

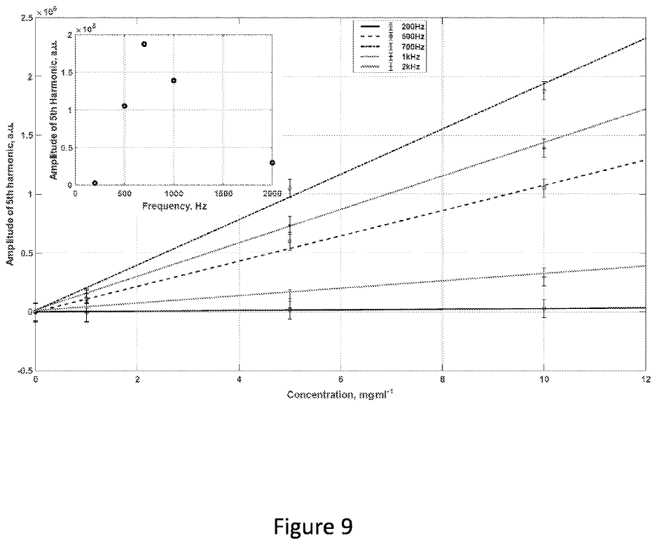

FIG. 9 shows a graph showing calibration series of aqueous suspensions of superparamagnetic iron oxide nanoparticles at (0, 1, 5 and 10) mg/ml conducted at different drive signal frequencies. Each frequency has a different but linear dependence on concentration but they are self-consistent demonstrating the use of the system 100 at different frequencies. Frequencies above and below the primary frequency of interest around 300 Hz are shown including 200 Hz, 500 Hz, 700 Hz, 1 kHz and 2 kHz. An insert is shown on the graph demonstrating the variability of the highest concentration of nanoparticles on frequency (the same data is shown in the main graph).

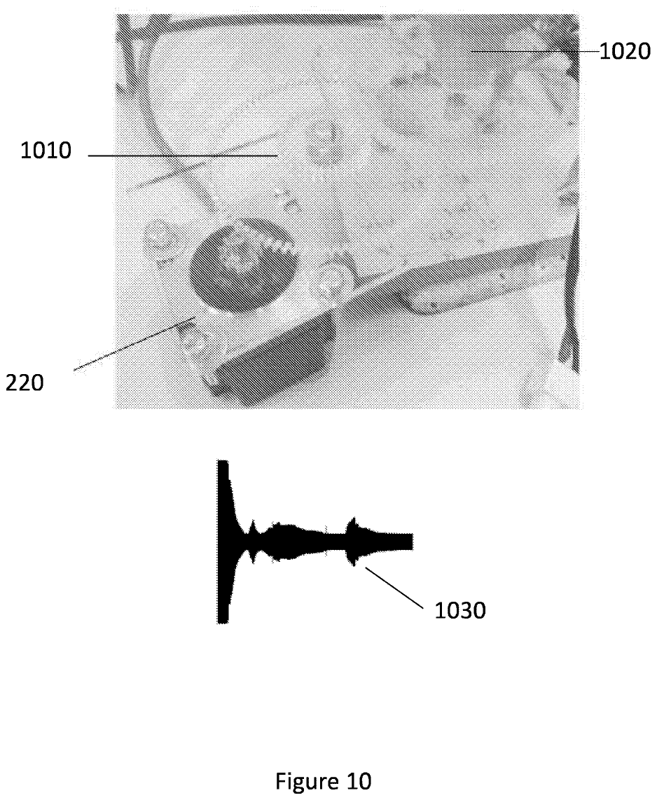

FIG. 10 shows part of an example Magnetic Particle Spectrometer system 100. A computer or microcontroller controlled stepper motor 220 is used to transfer torque through a drive train 1010 to the cancellation coil bobbin 1020. This bobbin 1020 has an external thread which is inserted into the main housing of the MPS probe. Rotating the stepper motor 220 spindle and thus the gear chain makes small rotational movements of the bobbin which cause a linear motion in (counterclockwise) or out (clockwise) of the drive coil 120 allowing the position to be fine tuned until it is in a corresponding field to the receiving coil 130. Since the coils are counterwound the same applied field will cause a cancellation when these two coils are connected in series. This process can be automated by the computer with the one or more processor(s) 201 and memory 201, where the memory 202 can store computer program instructions, which when executed by the processor(s) 201, is responsible for rotating the bobbin to find the minimum signal when no sample is present in the receiving coil 130. This is based on simple iterative search algorithm which takes the cancelation coil 150 past the optimum point and back again repeatedly until the signal is below the residual noise floor. An example of the signal present (amplified for clarity) throughout this process is also shown as an insert graph 1030.

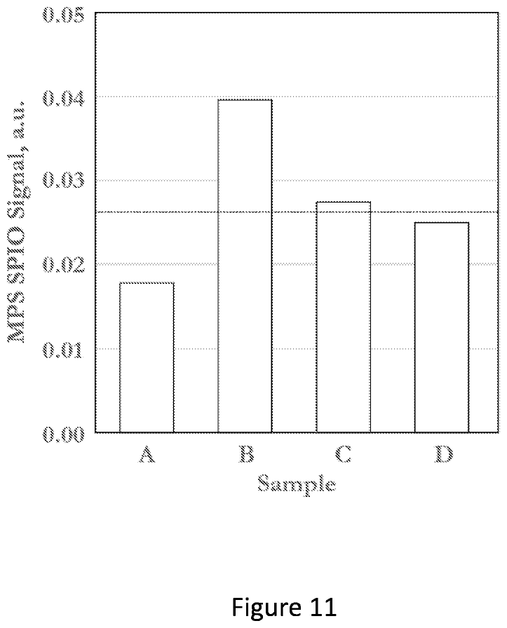

FIG. 11 shows an example of the use of the Magnetic Particle Spectrometer system 100 to accept or reject samples containing magnetic particles based upon meeting an appropriate threshold. In this example the labelling threshold was set to be around 0.026 a.u. Aqueous samples with different quantities of superparamagnetic iron oxide nanoparticle labels are then run with the system 100. Two which represent adequately labelled and two of which represent inadequately labelled. The plot shows that samples A and D do not meet the quality control standards whilst B and C do. C and D have concentrations such that they are just over and just under demonstrating the use of this technique as a quality control system.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method comprising:
placing a sample in proximity to a Magnetic Particle Spectrometer system, the Magnetic Particle Spectrometer system comprising a drive coil, a receiving coil;
wherein the sample comprises Magnetic Nanoparticles (MNPs) binded to at least one cell membrane of the sample, wherein the MNPs have a core diameter d_{c};
transmitting a magnetic field H(t) having a sinusoidally varying magnitude via a drive signal in the drive coil,
measuring a response magnetisation M(t) of the MNPs via an induced EMF in the receiving coil; and
determining a concentration c of the MNPs in the sample, by fitting a Langevin function to M(t) as a function of H(t).

2. A method as claimed in claim 1, wherein the Langevin function is:
$$M ( t ) = {m}_{s} c ( coth [ \frac{{m}_{s} {μ}_{0} H ( t )}{{k}_{B} T} ] - \frac{{k}_{B} T}{{m}_{s} {μ}_{0} H ( t )} ) , {m}_{s} = \frac{1}{6} π {d}_{c}^{3} {M}_{s} ,$$
where μ₀ is the permeability of free space, k_{B} is the Boltzmann constant and Mₛ is a known saturation magnetisation of the MNPs at a temperature T.

3. A method as claimed in claim 2, comprising:
determining the temperature T proximal to the Magnetic Particle Spectrometer system whilst the magnetic field H(t) is transmitted.

4. A method as claimed in claim 1, comprising comparing the determined concentration c against a concentration threshold to verify the sample.

5. A method as claimed in claim 1, wherein the Magnetic Particle Spectrometer comprises a cancellation coil, arranged to cancel out an induced emf in the receiving coil due to the drive signal.

6. A method as claimed in claim 1, comprising:
determining the concentration c of MNPs in multiple such samples; and
determining the variation in concentration c between the multiple samples.

7. A method as claimed in claim 1, wherein the drive signal is provided at less than 1 kHz.

8. A method as claimed in claim 1, wherein the drive signal is provided at less than 500 Hz; optionally the drive signal is provided at around 300 Hz.

9. A method as claimed in claim 1, wherein the sample comprises an assay.

10. A Magnetic Particle Spectrometer system comprising:
a drive coil, a receiving coil;
wherein the system is associated with a sample comprising Magnetic Nanoparticles (MNPs) binded to regions of at least one cell membrane of the sample, wherein the MNPs have a core diameter d_{c};
wherein the sample is located proximal to the Magnetic Particle Spectrometer system; and
wherein the system is arranged to:
transmit a magnetic field H(t) having a sinusoidally varying magnitude via a drive signal in the drive coil, and measure the response magnetisation M(t) of the MNPs via an induced EMF in the receiving coil; and
determine a concentration c of the MNPs in the sample, using a Langevin function.

11. A system as claimed in claim 10, wherein the Langevin function is:
$$M ( t ) = {m}_{s} c ( coth [ \frac{{m}_{s} {μ}_{0} H ( t )}{{k}_{B} T} ] - \frac{{k}_{B} T}{{m}_{s} {μ}_{0} H ( t )} ) , {m}_{s} = \frac{1}{6} π {d}_{c}^{3} {M}_{s} ,$$
where μ₀ is the permeability of free space, k_{B} is the Boltzmann constant and Mₛ is a known saturation magnetisation of the MNPs at a temperature T.

12. A system as claimed in claim 11, wherein the system is arranged to determine the temperature T proximal to the Magnetic Particle Spectrometer system whilst the magnetic field H(t) is transmitted.

13. A system as claimed in claim 10, wherein the Magnetic Particle Spectrometer system comprises a cancellation coil, arranged to cancel out an induced emf in the receiving coil due to the drive signal.

14. A system as claimed in claim 13, wherein the cancellation coil comprises a threaded surface, and the receiving coil comprises a threaded surface arranged to receive the cancellation coil via a threaded connection, wherein the position of the cancellation coil relative to the receiving coil is adjustable by rotating the cancellation coil relative to the receiving coil.

15. A system as claimed in claim 13, wherein the system is arranged to automatically adjust the position of the cancellation coil relative to the receiving coil using a closed feedback loop to optimally cancel out the induced emf in the receiving coil due to the drive signal.

16. A system as claimed in claim 10, wherein the drive signal is provided at less than 1 kHz.

17. A system as claimed in claim 10, wherein the drive signal is provided at less than 500 Hz; optionally the drive signal is provided at around 300 Hz.

18. A system as claimed in claim 10, wherein the sample comprises an assay.
